(19) United States
(12) Patent Application Publication
(10) Pub. No.: US 2017/0254167 A1
(43) Pub. Date: Sep. 7, 2017

(54) BLOWOUT PREVENTER PACKER INSERT

(71) Applicant: Hydril USA Distribution, LLC, Houston, TX (US)

(72) Inventor: William L. Carbaugh, Houston, TX (US)

(21) Appl. No.: 15/058,563

(22) Filed: Mar. 2, 2016

(51) Int. Cl. E21B 33/064; B29C 65/00; B29C 65/70; B29C 65/48

(52) U.S. Cl. CPC E21B 33/064; B29C 65/4835; B29C 66/742; B29C 65/70; B29K 2021/00

(57) ABSTRACT

A BOP packer, including a body disposed about a longitudinal axis and adapted to be compressed upon energization of the packer, and an insert having a form symmetrical about a plane parallel to the longitudinal axis, and embedded in the body with the body adhered to the insert to reduce extrusion of the body when the packer is energized. The insert includes an upper flange and a lower flange, each having a wedge-shaped configuration that expands from a relatively narrow portion at the end nearest the longitudinal axis, to a relatively wide portion at an opposite end, and a web element extending between the upper flange and the lower flange. The web element defines an elongated aperture through which a portion of the elastomeric body passes to help maintain the position of the metallic insert relative to the body, and to reduce shear forces between the insert and the body.

### BACKGROUND OF THE INVENTION

1. Field of the Invention

Embodiments disclosed herein relate generally to packers for use in oil field applications. In particular, embodiments disclosed herein relate to packers for use in annular blow out preventers.

2. Brief Description of Related Art

Blowout preventers (BOPS) are often employed in subsea oil and gas exploration. Such BOPs can be used to control pressures while drilling a well. Typically, BOPs include packers having inserts that provide structural support, reduce fatigue, and lessen weakening to the packers. Such fatigue and weakening can reduce the service life of the packer, or can result in fracture of the packer.

Another purpose of BOP packer inserts is to help reduce extrusion of the elastomer in the packers, which can be accomplished by embedding the inserts into the elastomer during the manufacturing process, and adhering the elastomer to surfaces of the insert. In some instances, however, the high shearing forces at the interface between the elastomer and the insert can cause the elastomer to separate from or slip relative to the insert.

### SUMMARY OF THE INVENTION

One embodiment of the present technology provides a blow-out preventer (BOP) packer, including an elastomeric body disposed at least partially about a longitudinal axis and adapted to be compressively displaced inwardly towards the longitudinal axis upon energization of the BOP packer, and a metallic insert having a form substantially symmetrical about a plane parallel to the longitudinal axis, and embedded in the elastomeric body with the elastomeric body adhered to the metallic insert to reduce extrusion of the elastomeric body when the BOP packer is energized. The metallic insert includes an upper flange and a lower flange, each of the upper and lower flanges having a substantially wedge shaped configuration that expands from a relatively narrow portion at the end nearest the longitudinal axis, to a relatively wide portion at an opposite end away from the longitudinal axis. The metallic insert further includes a web element extending between the upper flange and the lower flange, the web element defining a substantially elongated aperture through which a portion of the elastomeric body passes to help maintain the position of the metallic insert relative to the elastomeric body as the BOP packer is energized, and to reduce shear forces between the metallic insert and the elastomeric body along surfaces where the elastomeric body is adhered to the metallic insert.

Another embodiment of the present technology provides a BOP packer, including an elastomeric body disposed at least partially about a longitudinal axis and adapted to be compressively displaced inwardly towards the longitudinal axis upon energization of the BOP packer, and a plurality of metallic inserts embedded in the elastomeric body in substantially circumferentially spaced fashion in respective radial planes extending from the longitudinal axis of the elastomeric body, each of the metallic inserts having a form substantially symmetrical about a plane parallel to the longitudinal axis, and embedded in the elastomeric body with the elastomeric body adhered to the metallic insert to reduce extrusion of the elastomeric body when the BOP packer is energized. Each of the metallic inserts includes an upper flange and a lower flange, each of the upper flange and the lower flange having a substantially wedge shaped configuration that expands from a relatively narrow portion of the flange at the end of the flange nearest the longitudinal axis to a relatively wide portion of the flange at an opposite end away from the longitudinal axis. Each of the metallic inserts further includes a web element extending between the upper flange and the lower flange, the web element defining a substantially elongated aperture through which a portion of the elastomeric body passes to help maintain the position of the insert relative to the elastomeric body as the BOP packer is energized, and to reduce shear forces between the metallic insert and the elastomeric body along surfaces where the elastomeric body is adhered to the metallic insert.

Yet another embodiment of the present technology provides a method for limiting the extrusion of an elastomeric body in a BOP packer. The method includes the steps of embedding metallic inserts into the elastomeric body to provide rigid structure in the elastomeric body that resists extrusion during energization of the blowout packer, and adhering the elastomeric body to surfaces of the metallic inserts to limit extrusion of the elastomeric body around the metallic inserts. The method further includes the step of molding a portion of the elastomeric body through an elongated aperture in the metallic inserts to further reduce extrusion in the elastomeric body and reduce shear forces acting on the adhesive interface between the elastomeric body and the metallic inserts.

### BRIEF DESCRIPTION OF THE DRAWINGS

The present technology will be better understood on reading the following detailed description of nonlimiting embodiments thereof, and on examining the accompanying drawings, in which:

FIG. 1 shows a side schematic view of a lower marine riser package and a lower stack of a BOP assembly, including an annular BOP housing a BOP packer in accordance with an embodiment of the present technology;

FIG. 2 shows an isometric view of a BOP packer in accordance with an embodiment of the present technology.

FIG. 3 shows a side cross-sectional view of the blowout preventer packer of FIG. 2, taken along line 3-3 of FIG. 2;

FIG. 4 shows a side perspective view of a metallic insert in accordance with an embodiment of the present technology;

FIG. 5A shows an isometric view of an alternate embodiment of the an insert according to yet another embodiment of the present technology; and

FIG. 5B shows a side view of the embodiment of FIG. 5A.

### DETAILED DESCRIPTION OF THE PREFERRED EMBODIMENT

The foregoing aspects, features, and advantages of the present technology will be further appreciated when considered with reference to the following description of preferred embodiments and accompanying drawings, wherein like reference numerals represent like elements. The following is directed to various exemplary embodiments of the disclosure. The embodiments disclosed should not be interpreted, or otherwise used, as limiting the scope of the disclosure, including the claims. In addition, those having ordinary skill in the art will appreciate that the following description has broad application, and the discussion of any embodiment is meant only to be exemplary of that embodiment, and not intended to suggest that the scope of the disclosure, including the claims, is limited to that embodiment.

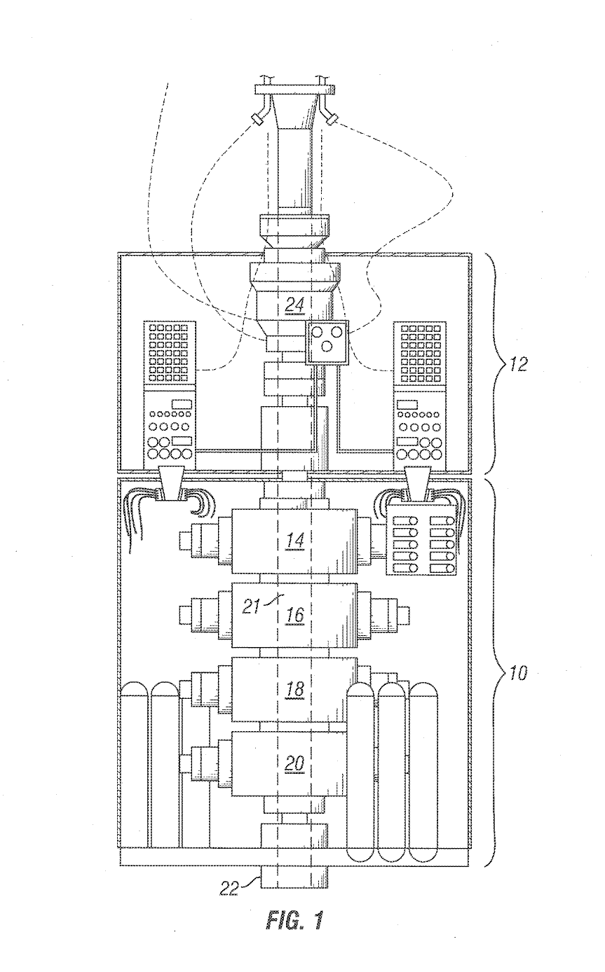

FIG. 1 shows a subsea blow out preventer (BOP) assembly, including a lower stack 10 and a lower marine riser package (LMRP) 12. Typically, the lower stack includes a series of stacked rams 14, 16, 18, 20. The lower stack 10 of FIG. 1, for example, can include a blind shear ram 14, a casing shear ram 16, and pipe rams 18, 20. In practice, the rams 14, 16, 18, 20 surround a bore 21 through which a drill pipe (not shown) passes. The lower stack 10 is positioned atop the wellhead 22, so that the drill pipe passes from the bottom of the lower stack 10 into the well through the wellhead 22. In addition, the LMRP 12 can include an annular BOP 24, which includes a BOP packer 100 (shown in FIG. 1). The purpose of the annular BOP and the rams is to control the well. For example, the BOP packer 100 inside the annular BOP can be energized so that it engages the drill pipe, and can control pressure in the annulus even as the pipe passes from the packer 100. In addition, if a surge of pressure develops in the well annulus, the BOP packer 100 inside the annular BOP 24 can be energized to seal against the drill pipe, thereby sealing the annulus. Thus, pressure below the annular BOP can be contained. The rams 14, 16, 18, 20 serve similar or related functions, typically below the annular BOP 24 in the lower stack 10.

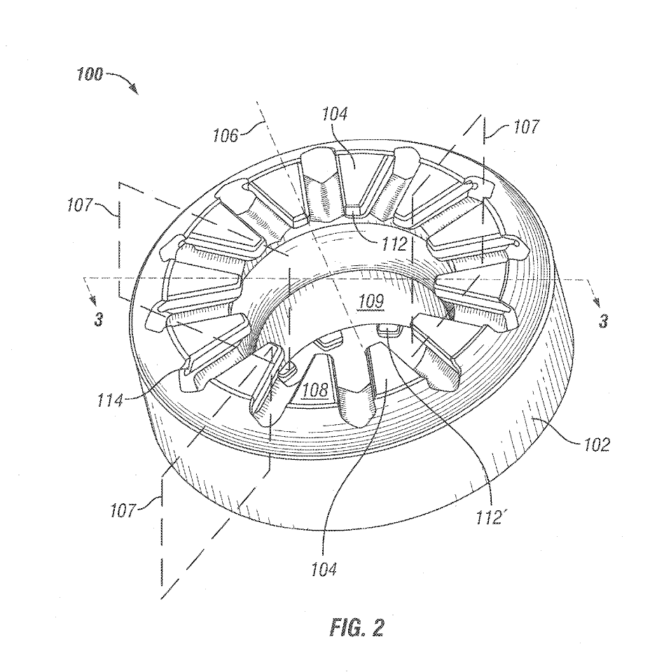

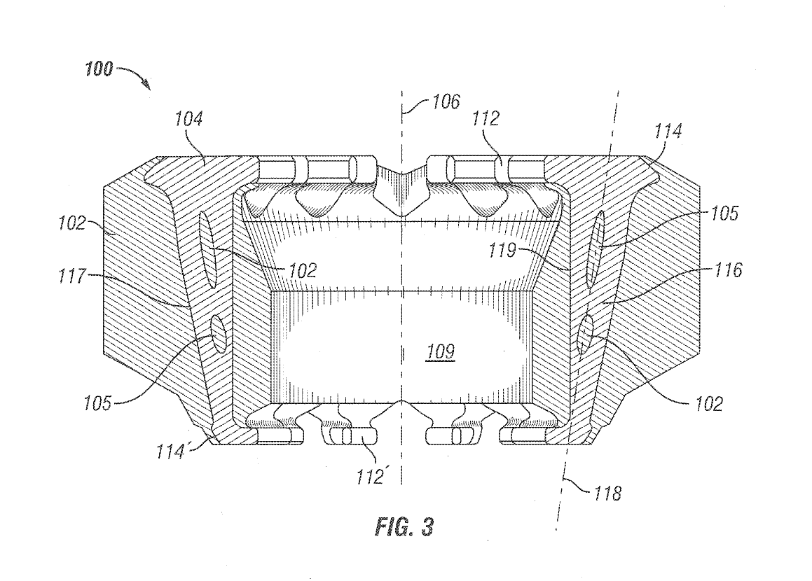

FIG. 2 depicts certain features of the BOP packer 100 that are helpful to an accurate description of the embodiments of the present technology, including an elastomeric body 102 and metal inserts 104. The metallic inserts 104 can be composed of any appropriate metal, such as, for example, alloy steel or Inconel. Similarly, the elastomeric body 102 can be composed of any appropriate elastomer, such as, for example, a nitrile elastomer, natural rubber, etc. The metal inserts 104 can each be bonded to the elastomeric body 102 using adhesive, such as, for example, a high strength epoxy adhesive. In some embodiments, the bond between the elastomeric body 102 and each metallic insert 104 may be along the width of the metallic insert 104. Alternately, or in addition to the adhesive bonding, the elastomeric body 102 may be mechanically attached to the metallic inserts 104. For example, the metallic inserts 104 can have elongated apertures 105 (shown in FIG. 3) through which a portion of the elastomeric body 102 passes. Embedding the metallic inserts 104 in the elastomeric body 102 of the packer 100, using such elongated apertures 105, may be accomplished during the manufacturing process, as described in greater detail below.

As shown in FIG. 2, the metallic inserts 104 may be disposed circumferentially in the packer 100 about a longitudinal axis 106, with each insert symmetrically aligned along a separate radial plane 107. When the packer is positioned in a BOP, the packer 100 surrounds the drill pipe, which passes through the packer 100 approximately along the longitudinal axis 106. When the BOP is activated to seal around the drill pipe, the packer 100 can be energized so that an inside surface 109 of the packer 100 compressively displaces inwardly towards the longitudinal axis 106 and into engagement with the drillpipe. As the packer 100 is energized, the metallic inserts 104 contract inwardly toward the drillpipe.

In some embodiments, the plurality of the metallic inserts 104 embedded in the elastomeric body 102 may be substantially equally spaced circumferentially around the packer 100. The substantially equal spacing of the metallic inserts 104 helps to equally distribute load stresses and reduce the presence of stress concentrations in the packer 100 when the packer 100 is energized. One benefit of reducing such stress concentrations in the present technology is greater durability of the BOP packer 100, and a greater ability to effectively function in extreme conditions. For example, the packer 100 of the present technology may be exposed to temperatures of up to about 350 degrees Fahrenheit (° F.) or more, and pressures of up to about 20,000 pounds per square inch (psi) or more.

Referring now to FIG. 3, there is shown a side cross-sectional view of the BOP packer 100 of FIG. 2, depicting the structure of metallic inserts 104 in the elastomeric body 102. Elongated apertures 105 are shown in the metallic inserts 104 with elastomeric material of the body 102 passing through the inserts 104. The apertures 105 help to ensure that the inserts 104 remain in a constant position relative to the elastomeric body 102, even as the packer 100 is energized and stresses develop between the elastomeric body 102 and the metallic inserts 104.

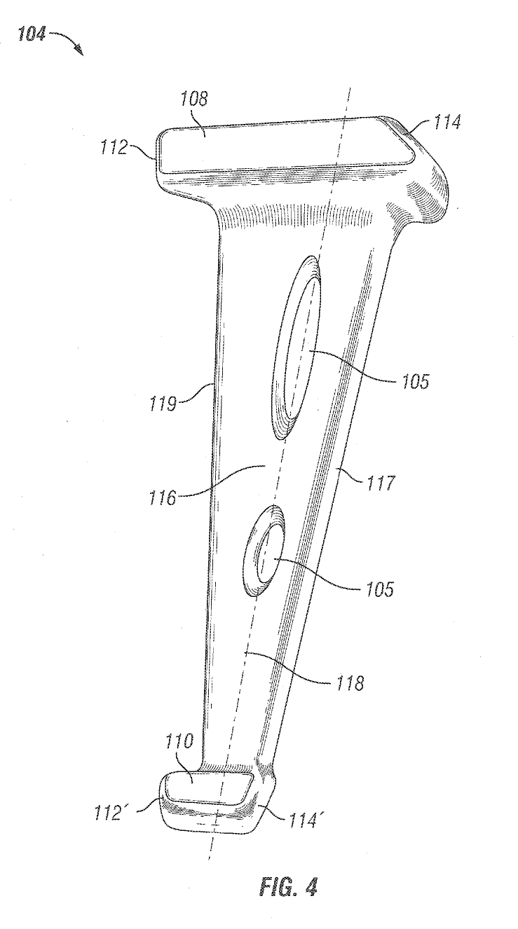

In FIG. 4, there is shown an enlarged perspective view of a metallic insert 104 in accordance with an embodiment of the present technology. As shown, the metallic insert 104 includes an upper flange 108 and a lower flange 110. The upper flange 108 has a substantially wedge shaped configuration that expands from a relatively narrow portion at the end 112 of the upper flange 108 nearest the longitudinal axis 106 of the packer 100, to a relatively wide portion of the upper flange 108 at an opposite end 114 away from the longitudinal axis 106. The wedge shape of the upper flange 108 is also depicted in FIG. 2. Similarly, the lower flange 110 also has a substantially wedge shaped configuration that expands from a relatively narrow portion at the end 112′ of the lower flange 110 nearest the longitudinal axis 106 of the packer 100, to a relatively wide portion of the lower flange 110 at an opposite end 114′. Also as shown in FIGS. 3 and 4, the end 114 of the upper flange 108 can be angled to increase the contact area between the elastomeric body 102 and the end 114 of the upper flange 108.

The metallic insert 104 further includes a web element 116 extending between the upper flange 108 and the lower flange 110. The web element 116 may be generally flat sided, and at least one edge 117 can be generally inclined at an angle relative to the longitudinal axis 106 (shown in FIGS. 2 and 3). In the embodiments shown, the web element has a smaller cross-sectional area than the upper flange 108 and the lower flange 110. In addition to angled edge 117, the web element 116 of the insert 104 can include an opposite edge 119.

The web element 116 of the metallic insert 104 includes at least one elongated aperture 105. One advantage of providing elongated apertures, as opposed to round apertures, is that elongated apertures allow for a maximization of the amount of elastomer that can pass through the aperture, while simultaneously minimizing the reduction of the web area. Thus, the strength and integrity of the web area is maintained while the benefits of the elastomeric body passing through the aperture are simultaneously realized.

The web elements 116 of the inserts 104 of FIGS. 3 and 4 are shown to have two apertures 105 of variable size and shape. In is to be understood, however, that any number of apertures of any shape can be included in the web element 116 consistent with the present technology. For example, in certain embodiments, the apertures can be oblong, oval, elliptical, tear-drop shaped, or egg-shaped. Similarly, the apertures 105 may be aligned along a plane 118 that is angled relative to the longitudinal axis 106 of the packer 100, although the angle of that plane can vary without departing from the scope of the present technology.

In some embodiments, edges 117, 119 of the web element 116 of the insert 114 have rounded profiles, as shown in FIG. 4. The rounded profiles provide a greater surface contact area between the metallic insert 104 and the elastomeric body 102 than would an edge with a straight profile, and the rounded profile helps to reduce stress concentrations at the interface between the metallic insert 104 and the elastomeric body 102. In addition, the rounded edges 117, 119 help to direct the flow of elastomer around the insert 104 during the manufacturing process, and to provide a better surface area to which the elastomer can bond, as compared to a sharp edge. In fact, in the embodiments of the inserts shown in the drawings, all edges of both the upper flange 108, 208, lower flange 110, 210, and web element 116, 216 have rounded profiles. While such a feature may not be included in all embodiments, it is helpful to reduce stress concentrations in the insert and increase surface area of the inserts.

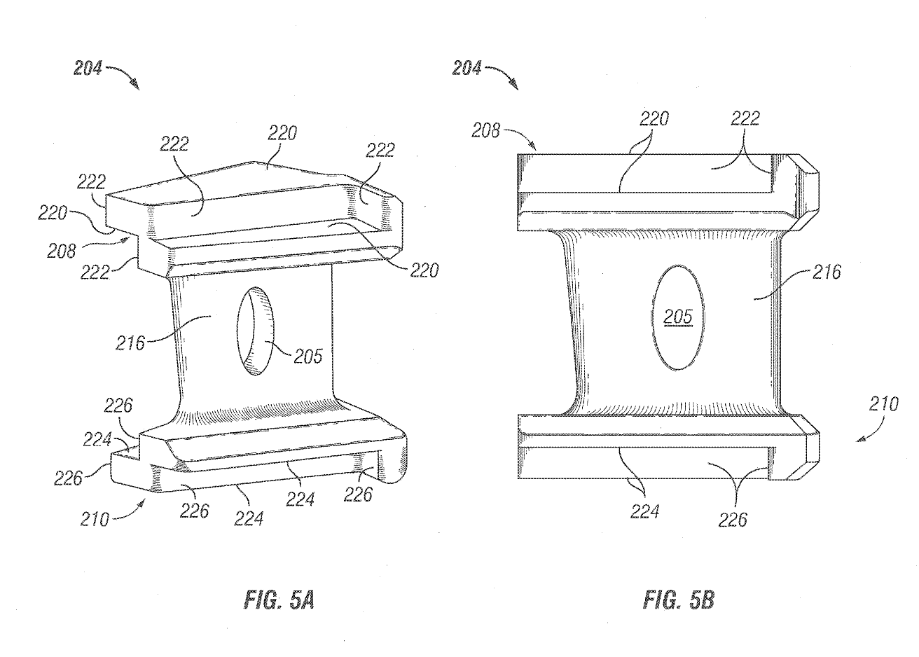

Referring now to FIG. 5A, there is shown an isometric view of an alternate embodiment of the metallic insert 204 including an upper flange 208 having a stepped configuration and a lower flange 210 having a stepped configuration. FIG. 5B depicts a side view of the metallic insert 204 of FIG. 5A. The stepped configuration of upper flange 208 includes multiple transverse surfaces 220 positioned in planes parallel, but axially offset from one another. The transverse surfaces 220 are interconnected by connecting surfaces 222 which, in the embodiments shown, are oriented in planes substantially perpendicular to the transverse surfaces 220. It is to be understood, however, that the connecting surfaces 222 need not be perpendicular to the transverse surfaces 220, but could alternately be angled relative to the transverse surfaces 220.

Similarly, the lower flange 210 includes multiple transverse surfaces 224 positioned in planes parallel, but axially offset from one another. The transverse surfaces 224 are interconnected by connecting surfaces 226 which, in the embodiments shown, are oriented in planes substantially perpendicular to the transverse surfaces 224. It is to be understood, however, that the connecting surfaces 226 need not be perpendicular to the transverse surfaces 224, but could alternately be angled relative to the transverse surfaces 224.

The stepped configuration of the upper flange 208 and the lower flange 210 in the embodiment of FIGS. 5A and 5B is advantageous because it provides an increased surface area for adhesion to the elastomeric body 102. In addition, the stepped structure of inserts 204 may help to eliminate potential extrusion gaps, and limit extrusion of the elastomeric body 102.

The inserts 204 shown in FIGS. 5A and 5B further include web element 216 connecting the upper flange 208 with the lower flange 210, and having an elongate aperture 205. The elongate aperture 205, as in the embodiments described above, provides a path for a portion of the elastomeric body 102 to pass through the insert 204 to help maintain the position of the insert 204 relative to the elastomeric body 102 and reduce stress loads on adhesive attaching the elastomeric body 102 to surfaces of the insert 204. Although a single aperture is shown in the figures, it is to be understood that any appropriate number of apertures can be included. In addition, although the shape of the aperture 205 is shown to be oval or elongated, in alternate embodiments, the shape can be oblong, elliptical, tear-drop shaped, egg-shaped, or any other appropriate shape.

A method of manufacturing the BOP packer 100 of the present technology includes cutting stock metal to create inserts 104, 204 using known machining methods. With the inserts cut, a manufacturer may cause them to undergo typical post machining procedures, such as deburring, polishing, etc. An adhesive, which may be, for example, a high strength epoxy adhesive, may then be applied to surfaces of the inserts 104, 204. In some processes, the application of epoxy can be accomplished by hand, using brushes, or sprayers. In other processes, the application of epoxy can alternately be accomplished by machine. In practice, the adhesive is activated by heat during the curing process (discussed below) in a vulcanization process.

The inserts 104, 204 are then positioned in a form, designed to hold the inserts in a predetermined position relative to one another while elastomer is wrapped around the inserts. The elastomer, which is generated using known techniques, is typically rolled into sheets, which can be cut and formed around the inserts into a desired configuration. Adhesive may be applied to the elastomer to further adhere the elastomer to the inserts. The elastomer-insert assembly is then introduced into a mold in a press. For packers used in annular packing units, the packers undergo a compression molding process. Alternately, for packers used in ram packers, the packers can undergo a transfer molding process.

The press can subject the elastomer-insert assembly to high pressure and, if desired, high temperature. Such high temperature cures the elastomer and activates the adhesives to bond the elastomer to the metallic inserts 104. The combination of temperature and pressure causes the elastomer to become viscous, and to flow and combine into a homogenous elastomeric body 102 that surrounds the inserts. During the process, the adhesive acts to bind the elastomer to the inserts 104, 204, and the elastomer flows through the apertures 105, 205 in the inserts to further bind the inserts to the elastomeric body 102. After pressing, the elastomer can be trimmed or cut as necessary to arrive at a finished BOP packer 100 according to the present technology.

Although the technology herein has been described with reference to particular embodiments, it is to be understood that these embodiments are merely illustrative of the principles and applications of the present technology. It is therefore to be understood that numerous modifications can be made to the illustrative embodiments and that other arrangements can be devised without departing from the spirit and scope of the present technology as defined by the appended claims.

What is claimed is:

1. A blow-out preventer (BOP) packer, comprising:
an elastomeric body disposed at least partially about a longitudinal axis and adapted to be compressively displaced inwardly towards the longitudinal axis upon energization of the BOP packer;
a metallic insert having a form substantially symmetrical about a plane parallel to the longitudinal axis, and embedded in the elastomeric body with the elastomeric body adhered to the metallic insert to reduce extrusion of the elastomeric body when the BOP packer is energized, the metallic insert comprising:
an upper flange and a lower flange, each of the upper and lower flanges having a substantially wedge shaped configuration that expands from a relatively narrow portion at the end nearest the longitudinal axis, to a relatively wide portion at an opposite end away from the longitudinal axis; and
a web element extending between the upper flange and the lower flange, the web element defining a substantially elongated aperture through which a portion of the elastomeric body passes to help maintain the position of the metallic insert relative to the elastomeric body as the BOP packer is energized, and to reduce shear forces between the metallic insert and the elastomeric body along surfaces where the elastomeric body is adhered to the metallic insert.

2. The BOP packer of claim 1, wherein the web element further comprises a plurality of substantially elongated apertures.

3. The BOP packer of claim 2, wherein the plurality of substantially elongated apertures are two apertures, and wherein the two apertures are aligned along a line that is angled relative to the longitudinal axis.

4. The BOP packer of claim 1, wherein the web element has a pair of edges, and wherein each edge has a rounded profile.

5. The BOP packer of claim 4, wherein all edges of the upper flange, lower flange, and web element have rounded profiles.

6. The BOP packer of claim 1, wherein the upper flange has a stepped transverse surface to increase the surface area of the upper flange.

7. The BOP packer of claim 6, wherein the lower flange has a stepped transverse surface to increase the surface area of the lower flange.

8. The BOP packer of claim 1, wherein the opposite end of the metallic insert away from the longitudinal axis is angled relative to the longitudinal axis to increase the surface area of the opposite end.

9. A blow-out preventer (BOP) packer, comprising:
an elastomeric body disposed at least partially about a longitudinal axis and adapted to be compressively displaced inwardly towards the longitudinal axis upon energization of the BOP packer; and
a plurality of metallic inserts embedded in the elastomeric body in substantially circumferentially spaced fashion in respective radial planes extending from the longitudinal axis of the elastomeric body, each of the metallic inserts having a form substantially symmetrical about a plane parallel to the longitudinal axis, and embedded in the elastomeric body with the elastomeric body adhered to the metallic insert to reduce extrusion of the elastomeric body when the BOP packer is energized, each of the metallic inserts comprising:
an upper flange and a lower flange, each of the upper flange and the lower flange having a substantially wedge shaped configuration that expands from a relatively narrow portion of the flange at the end of the flange nearest the longitudinal axis to a relatively wide portion of the flange at an opposite end away from the longitudinal axis;
a web element extending between the upper flange and the lower flange, the web element defining a substantially elongated aperture through which a portion of the elastomeric body passes to help maintain the position of the insert relative to the elastomeric body as the BOP packer is energized, and to reduce shear forces between the metallic insert and the elastomeric body along surfaces where the elastomeric body is adhered to the metallic insert.

10. The BOP packer of claim 9, wherein the plurality of the metallic inserts embedded in the elastomeric body are substantially equally spaced around the circumference of the BOP packer.

11. The BOP packer of claim 9, wherein the web element of each metallic insert further comprises a plurality of substantially elongated apertures.

12. The BOP packer of claim 11, wherein the plurality of substantially elongated apertures are two apertures, and wherein the two apertures are aligned along a line that is angled relative to the longitudinal axis.

13. The BOP packer of claim 9, wherein the web element of each metallic insert has a pair of edges, and wherein each edge has a rounded profile.

14. The BOP packer of claim 13, wherein all edges of the upper flange, lower flange, and web element of each metallic insert have rounded profiles.

15. The BOP packer of claim 9, wherein the upper flange of each metallic insert has a stepped transverse surface to increase the surface area of the upper flange.

16. The BOP packer of claim 15, wherein the lower flange of each metallic insert has a stepped transverse surface to increase the surface area of the lower flange.

17. The BOP packer of claim 9, wherein the opposite end of each metallic insert away from the longitudinal axis is angled relative to the longitudinal axis to increase the surface area of the opposite end.

18. A method for limiting the extrusion of an elastomeric body in a BOP packer, the method comprising:
(a) embedding metallic inserts into the elastomeric body to provide rigid structure in the elastomeric body that resists extrusion during energization of the blowout packer;
(b) adhering the elastomeric body to surfaces of the metallic inserts to limit extrusion of the elastomeric body around the metallic inserts;
(c) molding a portion of the elastomeric body through an elongated aperture in the metallic inserts to further reduce extrusion in the elastomeric body and reduce shear forces acting on the adhesive interface between the elastomeric body and the metallic inserts.

19. The method of claim 18, wherein step (b) further comprises applying adhesive to both the metallic inserts and the elastomeric body.

20. The method of claim 18, wherein step (c) further comprises molding a portion of the elastomeric body through a plurality of elongated apertures in each metallic insert to further reduce extrusion in the elastomeric body and reduce shear forces acting on the adhesive interface between the elastomeric body and the metallic inserts.
